(19) United States
(12) Patent Application Publication
(10) Pub. No.: US 2003/0233080 A1
(43) Pub. Date: Dec. 18, 2003

(54) Absorbent article with improved inconspicuousness

(76) Inventors: Lina Strand Backman, Goteborg (SE); Manoochehr Moslehi, Goteborg (SE); Urban Gabrielsson, Partille (SE)

Correspondence Address: BURNS, DOANE, SWECKER & MATHIS, L.L.P., P.O. Box 1404, Alexandria, VA, 22313-1404, US

(21) Appl. No.: 10/410,445

(22) Filed: Apr. 10, 2003

Related U.S. Application Data
(60) Provisional application No. 60/371,152, filed on Apr. 10, 2002.

(51) Int. Cl. A61F 13/15; A61F 13/20

(52) U.S. Cl. 604/385.01

(57) ABSTRACT

A part of an absorbent article (101, 201, 301, 401, 501, 601, 701, 801, 901, 1001) such as a sanitary towel, a panty liner or an incontinence pad or the like, has a regular transmittance in excess of 5% and a sheen of less than 25%.

### CROSS-REFERENCE TO RELATED APPLICATIONS

The present application claims the benefit of U.S. Provisional Application No. 60/371,152, filed in the United States on Apr. 10, 2002, the entire contents of which are hereby incorporated herein by reference.

### BACKGROUND OF THE APPLICATION

1. Technical Field

The present invention relates to an absorbent article such as a sanitary towel, a panty liner, an incontinence pad, or the like.

2. Background Art

There have been positive developments in terms of the comfort, reliability and inconspicuousness of absorbent articles such as diapers, incontinence pads and sanitary protection means.

It is particularly important that the users of sanitary protection means and/or incontinence pads feel that the articles they are wearing are inconspicuous, so that people in proximity to the user do not see that he or she is using the absorbent protector. A high degree of inconspicuousness has been able to be achieved, for example, by making the absorbent articles thinner and smaller in size, or more body-shaped. Articles have therefore not been able to be discerned through items of clothing such as tight-fitting trousers, figure-hugging dresses or the like.

Some absorbent articles have side tabs. The purpose of the side tabs is to be folded around the edges of the briefs in the crotch portion and in this way help to ensure a good fastening of the article in the briefs so that the article does not lie askew. An example of a document describing the importance of the side tabs for fastening of an absorbent article is EP 0,359,501. Another reason for providing an absorbent article with side tabs is that the risk of leakage occurring is reduced. Leakage often arises in articles without side tabs when the edges of the briefs in the crotch area become folded in over the top side of the article or when the article has ended up askew in the briefs. This usually happens when the article is narrower than the width of the briefs. Leakage can also occur when the body fluid cannot be absorbed in time by the central portion of the article but runs across the surface and over the side edges of the article where the bodily discharge comes into contact with the briefs. An article with side tabs secures the positioning of the article in the briefs and reduces the occurrence of leakage. In order to reduce the leakage along the edges of the article, the article is advantageously provided with side tabs which are as long as possible along the longitudinal side edges of the article. Articles with long side tabs are described, for example, in WO 98/25564. Articles without side tabs are therefore most often recommended for use when smaller flows of liquid are to be received by the article.

Many users consider an article with traditional side tabs to be insufficiently inconspicuous, because the side tabs are visible on the outside of the briefs, bathing costume, swimsuit or the like. This is of course more marked, the larger the side tabs are. An article which does not have side tabs is in this respect much more discreet because no side tabs have been folded around the leg openings of the briefs, bathing costume or swimsuit in the crotch area.

Manufacturers of sanitary articles have traditionally used material which has a whitish color in order to give the consumer a feeling of freshness with the article and in order to be suitable for white briefs, which in the past has been the most common color of briefs on the market. The market has now changed and there is a wide variety of briefs with both different colors and patterns, which means that articles with white side tabs or side tabs of a color different than that of the briefs are clearly visible on the outside of the briefs, bathing costume, swimsuit or the like.

Many users take part in sports which involve changing ones clothes in an environment which is to a greater or lesser extent public. In this connection, it is virtually impossible to conceal the use of a sanitary protection means with side tabs because these are visible on the outside of the briefs. The same problems arise in the case of activities such as swimming or sunbathing. There is also a requirement among some users of sanitary articles to be able to conceal the use of these in their home environment too. A further problem is that white side tabs can show through when thin white trousers are being worn, especially if the briefs have a pattern and/or a color which differs from that of the side tabs.

The above-described problems with articles which have side tabs have in part been solved by the side tabs being made of a transparent material. This is described, for example, in EP 1 078 619. The word “transparency” means that the transparent material or combination of materials has properties allowing visible light to pass through the material or combination of materials. Articles with transparent side tabs may permit improved inconspicuousness because the color of the item of clothing will be able to be discerned through the transparent side tabs.

In cases where the user is wearing briefs or a bathing suit, for example, made of a very thin or loosely woven fabric, the fact that the side tabs of the article are transparent is not enough to achieve sufficient inconspicuousness. Those parts of the article which come to lie between the briefs and the user's body may be discerned through the thin or loose fabric, because the articles which are at present commercially available have an absorbent core which is not transparent. The absorbent core of the article is usually made of cellulose fibers, synthetic fibers or similar fibers laid in layers or mixtures. Absorbent cores of the type in question here have no or extremely little transparency. Slightly greater transparency can be achieved if the absorbent core is made extremely thin, but not sufficiently great transparency for the desired inconspicuousness. The disadvantage of the abovementioned method of achieving increased transparency of the absorbent core is that the absorption capacity is reduced, that is to say the amount of liquid which the absorbent core can absorb in total is reduced, for which reason the article becomes unsuitable for certain purposes, for example for absorbing large amounts of liquid such as urine. Large amount of liquid is to be understood as meaning 50 ml in one discharge of liquid. To be able to produce thin absorbent cores, a certain amount of absorbent fiber material such as cellulose fluff, synthetic fibers or the like can be replaced by what is called superabsorbent material. Superabsorbent material is a material which can absorb very large amounts of liquid in relation to its weight and is a widely used material in the manufacture of sanitary articles such as baby diapers, incontinence pads, sanitary towels and panty liners. The superabsorbent material can consist, for example, of crosslinked polyacrylic acid, CMS (carboxymethyl starch), CMC (carboxymethyl cellulose), EHEC (ethylhydroxyethyl cellulose), HPC (hydroxypropyl cellulose), chitosan, alginate, pectin, xanthan or the like. Superabsorbent materials which consist in particular of a film have certain transparent properties. Examples of transparent superabsorbent materials are disclosed in the patent document EP 1 134 232.

Patent application EP 1 138 293 proposes that the article be designed so that it has fairly large surfaces which are transparent. The purpose of making the absorbent article transparent is to achieve a high degree of inconspicuousness for the person wearing the article, even in cases where the user is wearing items of clothing which have great transparency.

The articles described in applications EP 1 078 619 and EP 1 138 293 do not, however, achieve sufficient inconspicuousness. Simply defining a measure of transparency of the article does not completely satisfy the requirements of an inconspicuous article. None of the abovementioned documents describes the importance of the properties of the transparent surface of the article. In order to achieve a high degree of inconspicuousness of the article, the materials whose surfaces will be exposed to incident light should not reflect this light, because the reflected light is seen as a “gleam” on the surface.

This means that an article with very great transparency may nevertheless be discerned if incident light is allowed to be reflected on the surface of the article. This can apply to articles which have transparent side tabs or transparent articles without side tabs in cases where underclothes or other items of clothing placed nearest to the absorbent article are made of a loosely woven see-through fabric, for example lace material.

There may be circumstances where it is acceptable for the absorbent article to be of material with a high sheen, for example when the user's clothes also have a high sheen. Examples of textiles with a high sheen are types of polyester or silk.

However, these represent a minority of the underclothes which are available, and the invention is preferably intended to be used with other types of underclothes.

### OBJECTS AND SUMMARY

An object of the invention is to remedy the abovementioned problems and to produce an article with improved inconspicuousness.

An article of the type discussed in the introduction has been produced according to the present invention, which article substantially eliminates the problems that have been associated with previously known articles of this kind.

An embodiment of an absorbent article designed according to the invention is principally characterized by the fact that at least part of the absorbent article has a regular transmittance in excess of 5% and a sheen of less than 25%. For example, the article can have a regular transmittance in excess of 5% in at least the areas outside the longitudinal and transverse edges of the absorbent core, the material surfaces exposed to incident light according to this example having a sheen value of less than 25%.

By giving the transparent areas of the article a sheen of less than 25% and a regular transmittance in excess of 5%, it is possible to achieve improved conspicuousness for the user in cases where the user is wearing patterned and/or colored briefs or swimwear, or in cases where the item of clothing is made of thin, loosely made material.

The transparent areas preferably have a sheen of between 3 and 10%. A sheen of about 6% is most preferred. The sheen is measured on a complete material section. However, it is later possible, for example, to perforate or otherwise work the material. Perforation of a material reduces its sheen. A material with a sheen which is higher than acceptable can be chosen if it is later perforated or the surface structure is made more irregular. However, it is difficult to measure by this method on a material which has a large number of holes.

A material with a sheen which is higher than acceptable can be chosen if it is later perforated. The connection between the sheen of an unperforated material and the same material when perforated can be considered to be linear as a function of the starting sheen. If an unperforated material section has a sheen of one value and this material section is perforated, a sheen is thus obtained which is a product of the starting sheen and the open area of the perforated material section. That is to say, perforated material is also covered by the invention. As has been stated, the starting point is the sheen of an unperforated material section, which sheen is then reduced by the open area which the material section has when it is perforated. The value of the sheen of the perforated material can be described with the following equation:

Gₚ=Gₒ×f{(Aₜₒₜ−Aₕₒₗₑ)/Aₜₒₜ}

where

Gₚ=sheen of the perforated material

Gₒ=sheen of the unperforated material

f=a linear function

Aₜₒₜ=total area of the material section

Aₕₒₗₑ=total hole area

With a laminate comprising an upper perforated sheet and a lower sheet, it is a simple matter to measure the sheen.

In those cases where the transparent areas comprise several materials, the sheen of the whole laminate is measured. With a sheen of less than 25%, the material is felt to be invisible, assuming that the material is sufficiently transparent. An article with a sheen in excess of 25% would be considered to be too shiny.

A sheen of over 25% could be accepted, or might even be desirable, in cases where very shiny underwear is being used, for example, special grades of polyester or silk. However, this represents a small proportion of the presently available underclothes, and the invention is preferably intended to be used with other types of underclothes.

The word transparency is understood as a measure of the clearness of the material, that is to say how well a material can let incident light pass through the material, where the direction and the angle of the light let through correspond to the angle and direction of the incident light. The transmitted light (the light which has been let through) can exhibit a certain parallel displacement in relation to incident light, which is due, inter alia, to the thickness of the material and to how the light is refracted within the material layer. In physical terms, this is called “regular transmittance” and is defined by the physical formula described below:

R₁=(Rt_{light}/I_{light}) (%)

where

Rₜ=regular transmittance

Rt_{light}=regular transmitted light flux, i.e., the light which has passed through the material

I_{light}=incident light flux, i.e., the light flux which impacts the material.

A measure of the degree of regular transmittance is obtained by taking the quotient between the amount of transmitted light flux and the total amount of incident light flux, where the angle and the direction of transmitted light flux correspond to the angle and direction of the incident light flux.

The method used for the purpose is a standardized, accepted method which is well known in this field. The method has the reference ASTM D 1746-97. Regular transmitted light flux is measured on a complete material section, that is to say an unperforated material section.

When incident light impacts a material, there is, in addition to a regular transmission of the light, also a “diffuse scattering” of the incident light. This gives a measure of the material's “cloudiness”.

The physical description of “diffuse scattering” is the amount of transmitted light which has been scattered such that its direction deviates by more than a given angle from the incident light. Diffuse scattering in the material can be caused, for example, by an added substance with a refractive index different than that of the basic material, or by the fact that the material has a varying density in the direction of thickness of the material, or by the fact that the material's surface, on the side where the light leaves the material, has an uneven structure.

The calculations for the transparency of a material in the embodiments according to the invention will be covered only by values for the regular transmission. Hereinafter, the physical term “regular transmittance” will be called transparency.

The sheen of the material is understood as the specular reflection where the angle of reflection of the reflected light is the same as the angle of incidence of the incident light on the material.

When incident light impacts a material, there is, in addition to a “specular reflection”, also a “diffuse reflection” of incident light. The physical description of “diffuse reflection” is the reflected light which has an angle of reflection different than the angle of incidence of the incident light. Diffuse reflection of the material may be caused, for example, by irregularities in the surface of the material.

The sheen of a material in the embodiments according to the invention is calculated only on the basis of the “specular reflection”.

The “material's sheen” is obtained using the physical formula described below:

G=(S_{light}/I_{light}) (%)

where

G=the sheen of the material

S_{light}=specular reflection, i.e., the light flux which has been reflected off the material's surface at the same angle as the incident light.

I_{light}=incident light flux, i.e., the light flux which impacts the material.

Sheen is the quotient between the amount of reflected light flux and the total amount of incident light, where the angle of reflection of the reflected light flux is the same as the angle of incidence of the incident light flux.

The method used for the purpose is standard method ASTM D 2457-97. The method can be chosen to measure the sheen at an incident light flux with an angle of 20, 45 or 60°. In the measurements carried out in accordance with embodiments of the invention, the angle chosen was 60°.

By giving at least the material for those areas situated outside the absorbent core transparent properties and surface characteristics which prevent too much reflection of the incident light impacting the surface of the material, the inconspicuousness of the article can be greatly enhanced.

According to a preferred embodiment, the transparent areas of the article have a transmittance of at least 5%, preferably of at least 15%. An article with a transmittance of less than 5% may otherwise be considered too cloudy. Depending on what type of briefs or other clothes the article is placed in, and depending on the nature of the material of the article, it may be possible for the transparent areas of the article to be considered invisible at a sheen of less than 25%, even if the transmittance does not exceed 5%.

In order to enhance the inconspicuousness of the absorbent article, it is advantageous to limit the size of the absorbent core in cases where the latter is not sufficiently transparent. By limiting the size of the nontransparent core to the body openings for which the article is intended, for example introitus or urinary opening, the article does not show to any appreciable extent when the user is seen from straight in front or behind in cases where the user is wearing underwear made of thin and/or loosely woven material.

Even if the absorbent core is extremely small and only covers certain body openings, this does not greatly affect the level of reliability concerning leakages, since the material outside the absorbent core has been designed as a safety zone which can collect a small amount of liquid in cases where the absorbent core has not been able to absorb the entire amount of liquid because of the discharge of liquid onto the article taking place too quickly or because of the absorbent core having been incorrectly positioned. The absorbent core must of course be constructed in such a way that it covers the absorption requirements for which the article is designed.

According to a preferred embodiment of the invention, a second separate sheet is placed on the absorbent core's second surface which is intended to be directed away from the user's body during use. The second separate sheet in this case extends outside the longitudinal and transverse edges of the absorbent core of the article.

The second separate sheet in this case comprises a liquid-tight sheet with a regular transmittance in excess of 5% and a sheen of less than 25% in at least the areas outside the absorbent core of the article.

The separate second sheet can, for example, be colored, provided with a color print or embossed in such a way that the material becomes less transparent in the area of the absorbent core. Such treatment may be desirable given that some users find it off-putting to think that the core of the absorbent article can be seen. This problem is particularly pronounced when the absorbed body fluid is red-colored menstrual fluid.

Of course, treatment of the second separate sheet in the area of the absorbent core only applies if there is no need for the area of the absorbent core to have transparent properties.

According to another embodiment of the invention, the second separate sheet which extends outside the longitudinal and transverse edges of the absorbent core of the article, placed on the absorption core's second surface which is intended to be directed away from the user during use, comprises a liquid-permeable sheet.

The second separate sheet has at least a regular transmittance in excess of 5% and has a sheen of less than 25% in at least the areas outside the absorbent core of the article.

This embodiment has been provided with a hydrophobic fastening adhesive on that side of the second separate sheet directed away from the absorbent core. The hydrophobic fastening adhesive covers at least the area of the absorbent core in order thereby to ensure that liquid reaching the absorbent core does not penetrate through the second separate sheet and thus cause stains to appear on the user's clothes.

If it is desired that the article also has sufficient transparency in the area inside the edges of the absorbent core, then it is preferable not only for the second separate sheet to be transparent, but for the absorbent core and the fastening adhesive to be transparent too.

According to a further embodiment of the invention, the second separate sheet comprises a liquid-permeable sheet. The second separate sheet is placed on the absorbent core's second surface which is intended to be directed away from the user's body during use. The second separate sheet extends outside the longitudinal and transverse edges of the absorbent core of the article.

The second separate sheet has at least a regular transmittance in excess of 5% and has a sheen of less than 25% in at least the areas outside the absorbent core of the article.

This embodiment has been provided with a liquid barrier sheet placed against that side of the separate liquid-permeable second sheet directed towards the absorbent core. The liquid barrier sheet will cover the area of the absorbent core. In this way, the second separate sheet comes to be liquid-tight within the area of the absorbent core.

According to yet another embodiment of the invention, the second separate sheet which extends outside the longitudinal and transverse edges of the absorbent core of the article is placed on the absorbent core's second surface intended to be directed away from the user's body during use of a liquid-permeable sheet.

The second separate sheet has at least a regular transmittance in excess of 5% and has a sheen of less than 25% in at least the areas outside the absorbent core of the article. This embodiment has been provided with a liquid barrier sheet placed against that side of the second separate sheet directed away from the absorbent core. The liquid barrier sheet will cover the area of the absorbent core, so that the second separate sheet comes to have liquid-tight properties within the area of the absorbent core.

According to another embodiment, the article has been provided with a first separate sheet which extends outside the longitudinal and transverse edges of the absorbent core of the article, placed on the absorption core's first surface which is intended to be directed towards the user's body during use.

The separate material is liquid-permeable in the area of the absorbent core and has a regular transmittance in excess of 5% and a sheen of less than 25% in at least the areas outside the longitudinal and transverse edges of the absorbent core of the article.

The first separate sheet placed on the first surface of the absorbent core is liquid-tight and, in the area of the absorbent core, has been provided with a plurality of openings to allow the arriving liquid to run down to the absorbent core lying underneath. The absorbent core has been provided with a hydrophobic adhesive fastener on the side which is intended to be directed towards the user's underwear during use. The hydrophobic adhesive fastener will cover at least the area of the absorbent core and in this way function as a liquid-tight sheet which prevents arriving liquid from penetrating through the article. As an alternative to the hydrophobic fastener, it is of course possible to provide the article with an extra sheet which is liquid-tight in a similar way to those previously described in one of the abovementioned illustrative embodiments.

According to a further embodiment, the first separate sheet has been designed with one large opening, instead of the plurality of small openings as before.

According to another embodiment, the article has been provided with a first separate sheet which extends outside the longitudinal and transverse edges of the absorbent core of the article, the sheet being placed on the absorption core's first surface which is intended to be directed towards the user's body during use. The first separate sheet has at least a regular transmittance in excess of 5% in the areas outside the longitudinal and transverse edges of the absorbent core of the article and has a sheen of less than 25%.

The first separate sheet in this case comprises a liquid-permeable sheet. The article according to this embodiment has been provided with a liquid barrier sheet placed against that surface of the absorbent core which, during use, is intended to be directed towards the user's underwear. The liquid barrier sheet will at least cover the area of the absorbent core in order to ensure that arriving liquid does not leak through and thus soil the user's underwear.

According to yet another embodiment, the article has been provided with a first separate sheet placed on the absorbent core's first surface, and with a second separate sheet placed on the absorbent core's second surface, said sheets extending outside the longitudinal and transverse edges of the absorbent core. The first separate sheet and the second separate sheet are connected to each other outside the absorbent core by means of a seal. The separate materials have a regular transmittance in excess of 5% at least in the areas outside the longitudinal and transverse edges of the core of the absorbent article and have a sheen of less than 25%. The first separate sheet, which is placed on the absorbent core's first surface, is in this case liquid-permeable, and the second separate sheet, which is placed on the absorbent core's second surface, is liquid-tight.

According to yet another embodiment, the first and second separate sheets which cover the first surface and second surface of the absorbent core extend further outside the longitudinal edges of the core so that side tabs are formed. These side tabs are of such a shape and size that, during use of the article, they can be folded around the leg edges in the crotch portion of a pair of briefs.

According to yet another embodiment, the article has been provided with a first separate sheet placed on the absorbent core's first surface, and with a second separate sheet placed on the absorbent core's second surface. The extent of the first separate sheet and of the second separate sheet corresponds to the extent of the absorbent core. All the materials included in the article have a regular transmittance in excess of 5% and a sheen of less than 25%.

The first separate sheet, which is placed on the absorbent core's first surface, is in this case liquid-permeable, and the second separate sheet, which is placed on the absorbent core's second surface, is liquid-tight.

In the described embodiments according to the invention, the sheets of material included in the article are fastened to one another by transparent adhesive, by ultrasound techniques or the like. A possible example of transparent adhesive for connecting different sheets to one another is a structural adhesive of the Dispomelt 2000 type produced by National S & C.

### BRIEF DESCRIPTION OF THE DRAWINGS

The embodiments of the invention will be described in greater detail below with reference to the figures which are shown in the attached drawings.

FIG. 1 shows a sanitary towel, seen from the side which will be directed towards the user during use;

FIG. 2 shows a section along the line II-II through the sanitary towel in FIG. 1;

FIG. 3 shows a longitudinal section along the line III-III through the sanitary towel in FIG. 1;

FIG. 4 shows the sanitary towel from FIGS. 1-3 with an alternative fastener arranged on the underside of the towel, seen from the side which will be directed towards the user during use;

FIG. 5 shows a section along the line V-V through the sanitary towel in FIG. 4;

FIG. 6 shows a longitudinal section along the line VI-VI through the sanitary towel in FIG. 4;

FIG. 7 shows a sanitary towel according to another embodiment of the invention, seen from the side which will be directed towards the user during use;

FIG. 8 shows a section along the line VIII-VIII through the sanitary towel in FIG. 7;

FIG. 9 shows a longitudinal section VIV-VIV through the sanitary towel in FIG. 7;

FIG. 10 shows a sanitary towel according to another embodiment of the invention, seen from the side which will be directed towards the user during use;

FIG. 11 shows a section along the line XI-XI through the sanitary towel in FIG. 10;

FIG. 12 shows a longitudinal section along the line XII-XII through the sanitary towel in FIG. 10;

FIG. 13 shows a sanitary towel according to another embodiment of the invention, seen from the side which will be directed towards the user during use;

FIG. 14 shows a section along the line XIV-XIV through the sanitary towel in FIG. 13;

FIG. 15 shows a longitudinal section XV-XV through the sanitary towel in FIG. 13;

FIG. 16 shows a sanitary towel according to another embodiment of the invention, seen from the side which will be directed towards the user during use;

FIG. 17 shows a section along the line XVII-XVII through the sanitary towel in FIG. 16;

FIG. 18 shows a longitudinal section along the line XVIII-XVIII through the sanitary towel in FIG. 16;

FIG. 19 shows a sanitary towel according to another embodiment of the invention, seen from the side which will be directed towards the user during use;

FIG. 20 shows a section along the line XX-XX through the sanitary towel in FIG. 19;

FIG. 21 shows a longitudinal section along the line XXI-XXI through the sanitary towel in FIG. 19;

FIG. 22 shows a sanitary towel according to another embodiment of the invention, seen from the side which will be directed towards the user during use;

FIG. 23 shows a section along the line XXIII-XXIII through the sanitary towel in FIG. 22;

FIG. 24 shows a longitudinal section along the line XXIV-XXIV through the sanitary towel in FIG. 22;

FIG. 25 shows a sanitary towel according to another embodiment of the invention, seen from the side which will be directed towards the user during use;

FIG. 26 shows a section along the line XXVI-XXVI through the sanitary towel in FIG. 25;

FIG. 27 shows a longitudinal section along the line XXVII-XXVII through the sanitary towel in FIG. 25;

FIG. 28 shows a sanitary towel according to another embodiment of the invention, seen from the side which will be directed towards the user during use;

FIG. 29 shows a section along the line XXVIII-XXVIII through the sanitary towel in FIG. 28; and

FIG. 30 shows a longitudinal section along the line XXVIV-XXVIV through the sanitary towel in FIG. 28.

### DETAILED DESCRIPTION OF THE PREFERRED EMBODIMENTS

In the examples, absorbent articles in the form of sanitary towels are described, but the invention can of course be applied to panty liners, incontinence pads, or the like.

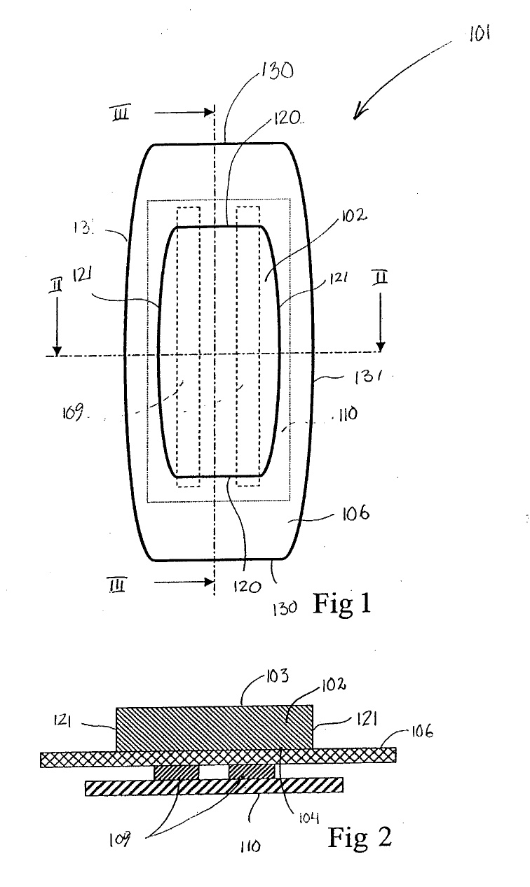

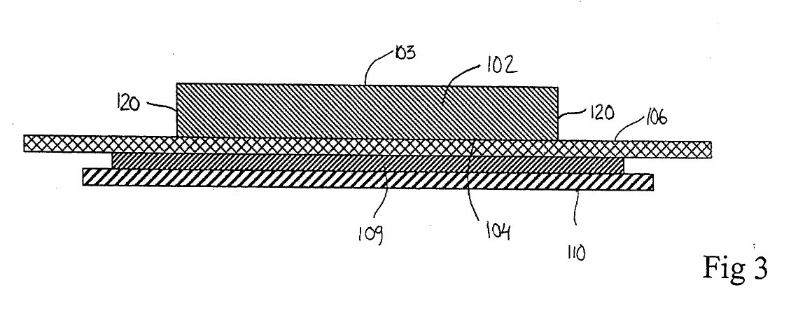

FIGS. 1-3 show an embodiment of a sanitary towel 101 which has two transverse edges 130, and two longitudinal edges 131. The sanitary towel in FIGS. 1-3 has a substantially rectangular shape, but it can also be designed so that it has an hourglass shape, a round shape, a trapezoid shape, or another shape suitable for the purpose. The sanitary towel can also be made three-dimensional, that is to say the surface of the sanitary towel which will be directed towards the user is of such a shape that it is adapted to the user's anatomy. Sanitary towels which have a three-dimensional shape are often designed with a ridge-like elevation, referred to as a hump, so as to be adapted to the genitals, introitus or between the buttocks.

The sanitary towel comprises an absorbent core 102 which can comprise one or more sheets of material. In cases where there is no particular requirement in respect of transparency or the sheen value of the sanitary towel in the area of the absorbent core, nontransparent absorbent materials can advantageously be chosen. Suitable nontransparent materials for the absorbent core 102 include, for example, cellulose pulp.

Fluff pulp is usually supplied as rolls, bales or sheets which are dry-defiberd and converted in fluffed form into a pulp mat. Superabsorbents are also generally admixed. Superabsorbents are polymers with the capacity to absorb several times their own weight of water or body fluid. Examples of other usable materials are various types of regenerated cellulose, for example, viscose fibers as described in SE 9903070-2. Natural fibers such as cotton fibers, peat or the like can also be used. It is of course also possible to use absorbent and/or hydrophilic synthetic fibers or mixtures of natural fibers and synthetic fibers. Other examples of materials which can be used for the absorbent core are, for example, foamed materials having an open cell structure.

If the sanitary towel is to have transparency in the area of the absorbent core, it is of course necessary for the absorbent core to be made of a transparent material. Examples of a transparent superabsorbent material are set out in the patent document EP 1,134,232.

The absorbent core 102 also has two surfaces, namely a first surface 103 which is directed upwards towards the user and a second surface 104 which is directed away from the user during use.

To obtain a soft and comfortable surface of the sanitary towel 101, it is preferable for the first surface 103 of the absorbent core to be soft and comfortable. If the absorbent core 102 consists of a loosely bound fiber structure with or without unbound superabsorbent particles, the absorbent core 102 may crease and be “crumpled up” during use and thus become very uncomfortable. An unbound absorbent core 102 can advantageously be covered or enclosed so that at least the first surface 103 of the absorbent core comprises a liquid-permeable sheet which is not appreciably affected by mechanical wear caused by the user in the dry and wet states. A sheet whose purpose is to provide good comfort for the user is often referred to in this field as a comfort layer. The comfort layer is not depicted in the figures but will constitute an obvious alternative for those embodiments in which the first surface 103 of the absorbent core lies directly against the user and where the absorbent core 102 is constructed in such a way that an extra comfort sheet is required to maintain good comfort for the user during use.

A comfortable surface structure for the first surface 103 of the absorbent core can be obtained, for example, by providing said surface with a sheet of fibrous web comprising absorbent or nonabsorbent fibers of cotton, polyethylene, polypropylene or the like. The fibrous web is advantageously bonded to the underlying material of the absorbent core 102, for example, by a mechanical pressing technique which results in the fibers of the fibrous web and of the absorbent core 102 becoming entangled in one another, or by applying energy so that a certain fusing of the fibrous web and/or fibers in the absorbent core 102 takes place and the fibrous web can adhere to the absorbent core 102. Other examples of usable comfort sheets placed on the first surface 103 of the absorbent core are bonded nonwoven sheets, three-dimensional plastic sheets, tissue sheets, knitted material, woven material or the like.

If it is desired that the sanitary towel 101 has transparency in the area of the absorbent core, then, in addition to the transparent absorbent core 102, it is necessary to choose a comfort sheet which has transparent properties. Examples of materials for transparent comfort sheets are three-dimensional liquid-permeable natural plastic film or bonded fiber material comprising natural fibers of polyethylene or polypropylene. The word natural means films or fibers without added organic or inorganic pigments, for example titanium dioxide.

When the absorbent core 102 comprises a foam material, tissue material, low-density airlaid, crocheted or knitted material, carded material, gel, hydrogel, combinations of these materials or similar materials with partially or completely bonded surface structure, the absorbent core 102 does not have to be provided with an extra comfort layer on the side of the first surface 103 of the absorbent core.

The sanitary towel 101 also comprises a second separate sheet 106 placed in such a way that it covers the second surface 104 of the absorbent core 102 and extends outside the longitudinal and transverse edges 121, 120 of the absorbent core. The second separate sheet 106 is characterized in that it is at least transparent in the areas outside the longitudinal edges 121 and transverse edges 120 of the absorbent core 102.

The second separate sheet 106 shown in the illustrative embodiment in FIGS. 1-3 comprises a thin, liquid-tight transparent flexible plastic film. Examples of suitable films are natural polypropylene and polyethylene films or laminates of these. The word natural means films without added organic or inorganic pigments, for example titanium dioxide.

To increase user comfort, the side of said second separate sheet 106 directed towards the user's body, can be treated for example with an embossed pattern. An embossed structure of the second separate sheet 106 feels less tacky to the user than if the second separate sheet 106 has a smooth surface structure.

An embossed pattern can be obtained by mechanical treatment, for example, with the second separate sheet 106 being guided through a nip between two rollers, where one of the rollers is designed in such a way that the second separate sheet 106 acquires an embossed surface.

It is also advantageous if the second separate sheet 106 is able to breathe, that is to say is able to let moist air through (but not liquid), as this increases user comfort. The second separate sheet 106 has a regular transmittance in excess of 5% and a sheen of less than 25%.

Some users find a visible core 102 off-putting, especially when the absorbed fluid is colored menstrual fluid. Therefore, in those cases where the sanitary towel 101 does not necessarily need to have transparent properties in the area of the absorbent core 102, it is possible to treat the second separate sheet 106 in the area of the absorbent core 102 in such a way as to obtain a reduced visibility of the second surface 104 of the absorbent core. Increased opacity can be obtained in the second separate sheet 106 by means of the plastic film being colored with titanium dioxide, for example, or by means of the surface being embossed. This is not illustrated in FIGS. 1-3.

A fastener 109 in the form of two longitudinal adhesive strips is arranged in the area of the absorbent core 102, on that side of the second separate sheet 106 intended to be directed away from the user during use. The fastener 109 is protected by a removable protective sheet 110. The protective sheet 110 is intended to protect the adhesive fastener 109 from dirt and dust, and from adhering to other undesired surfaces or to itself, until the sanitary towel is to be used. As an alternative to the adhesive fastener 109, it is possible to use means which have mechanical adherence or high friction. Examples of an alternative fastener 109 are velcro material or foamed material. When a velcro material or foamed material is used as a fastener 109, the protective sheet 110 can be omitted. The fastener 109 preferably comprises a transparent fastening adhesive which is protected by a removable protective sheet 110.

If it is desired that the sanitary towel has transparency in the area of the absorbent core 102, then, in addition to the absorbent core 102 and the second separate material sheet 106 being transparent, it is also necessary for the fastener 109 to have transparent properties. Examples of suitable transparent fastener 109 are adhesives without admixture of pigments, for example, Ecomelt H 145 produced by Collano.

An alternative way of reducing the visibility of the second surface 104 of the absorbent core 102 is for the above-described fastener 109 to be chosen such that it covers a greater part of the extent of the absorbent core 102 and for the material of the fastener 109 to be opaque. Here, opaque signifies that the material has such properties that the material lying underneath is not visible through the opaque material. Examples of opaque fastener 109 can be, for example, adhesives containing coloring pigments, mechanical fastener 109 such as velcro or foamed material, or the like.

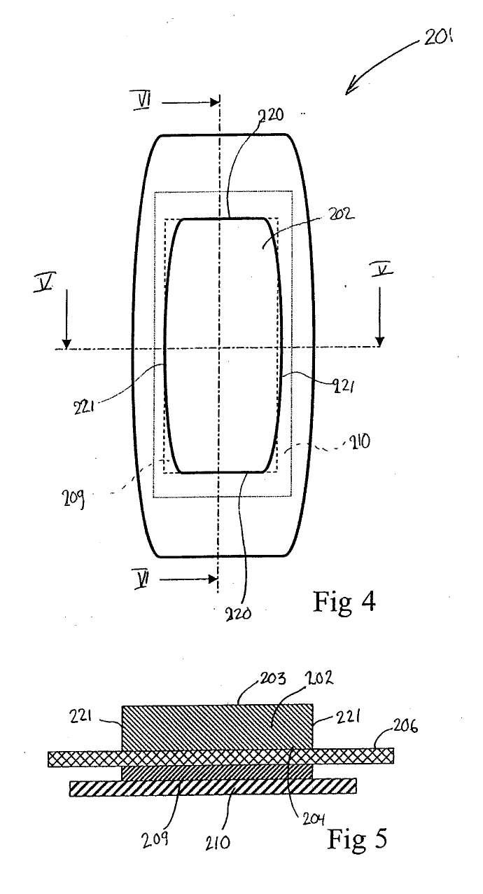

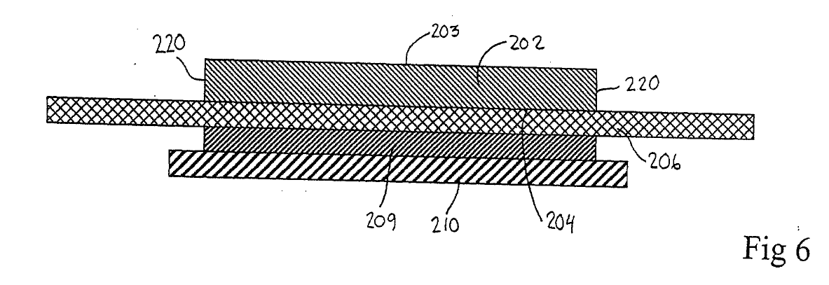

FIGS. 4-6 show an embodiment of a sanitary towel 201 of substantially the same structure as the sanitary towel 101 in FIGS. 1-3. The numbering of the various parts of the sanitary towel in FIGS. 4-6 is therefore analogous to the numbering used in the description of the sanitary towel 101 in FIGS. 1-3. However, the sanitary towel 201 shown in FIG. 4 differs in two respects from the previously described sanitary towel 101.

The first difference is that the second separate material sheet 206 placed against the second surface 204 of the absorbent core is liquid-permeable. The second separate sheet 206 has transparent properties at least in the areas which extend outside the absorbent core 202. The second separate sheet 206 preferably comprises liquid-permeable material of nonwoven fiber fabric, called nonwoven material.

Another example of a liquid-permeable material is a sheet of continuous longitudinal bonded fibers. Other possible liquid-permeable materials are perforated plastic films, net, knitted or woven textiles and combinations and laminates of the material types listed. The material consists of natural fibers of polyethylene or polypropylene without added organic or inorganic pigments, for example titanium dioxide, in order to obtain transparent properties.

The second difference between the sanitary towel 201 and the previously described sanitary towel 101 in FIGS. 1-3 is that the fastener 209 covers at least the extent of the core 202 and the coverage it provides is such that liquid reaching it cannot penetrate through the fastener 209 and risk causing stains on the user's clothes. Suitable liquid-tight fasteners are foamed material in which at least one surface has closed cells, hydrophobic adhesive or the like. When adhesive is used as a fastener 209, it is important for the second separate sheet 206 to be coated so that the adhesive sheet does not have openings large enough for the absorbed liquid to penetrate through the adhesive sheet 209.

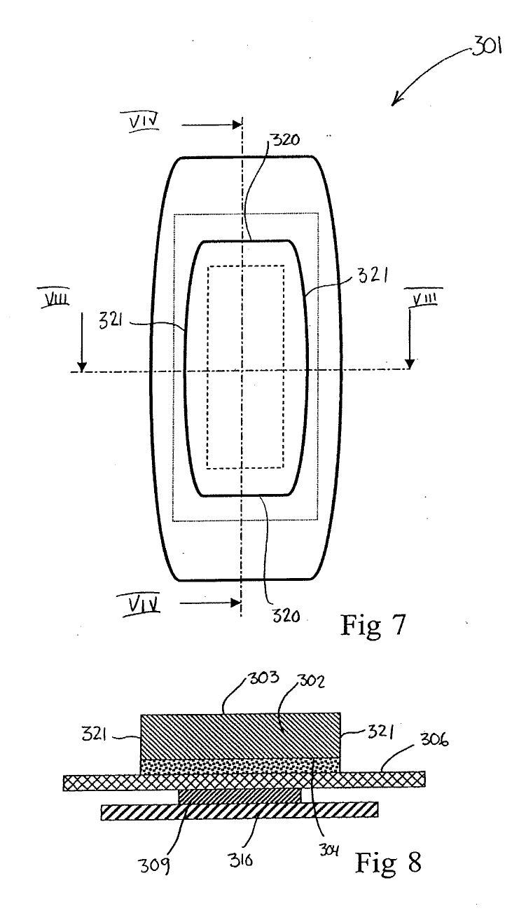

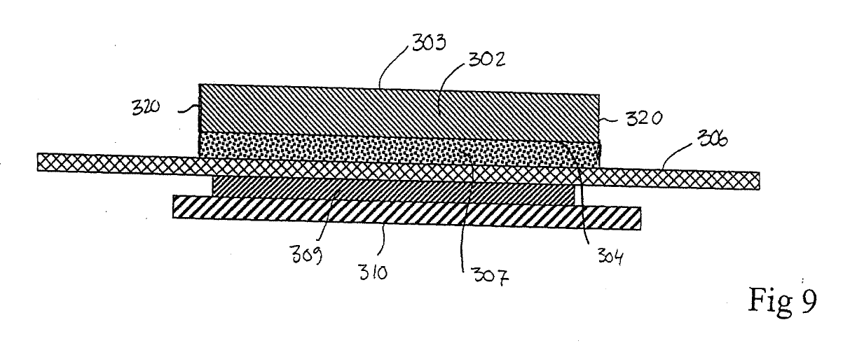

FIGS. 7-9 show an embodiment of a sanitary towel 301 of substantially the same structure as the sanitary towel 201 in FIGS. 4-6. The numbering of the various parts of the sanitary towel in FIGS. 7-9 is therefore analogous to the numbering used in the description of the sanitary towel 201 in FIGS. 4-6.

However, the sanitary towel 301 shown in FIG. 7 differs in one respect from the previously described sanitary towel 201.

To ensure that liquid reaching the sanitary towel 301 does not penetrate through the liquid-permeable second separate sheet 306 and thus cause staining of the user's clothes, the sanitary towel 301 is provided with a liquid barrier sheet 307 placed against that surface of the second separate sheet 306 which is intended to be directed towards the second surface 304 of the absorbent core. The liquid barrier sheet 307 has a greater extent than or covers at least the same extent as the absorbent core 302. The liquid barrier sheet 307 consists, for example, of hydrophobic adhesive, plastic film, foamed material with at least one surface of closed cells, hydrophilic fiber structures such as nonwoven, cellulose fiber sheets which have been treated with a hydrophobicizing means so that these have a surface which is not liquid-permeable.

For the sanitary towel 301, there is no requirement for the fastener 309 to have a special configuration or be liquid-tight, since the liquid barrier sheet 307 prevents liquid from spreading further to the fastener 309.

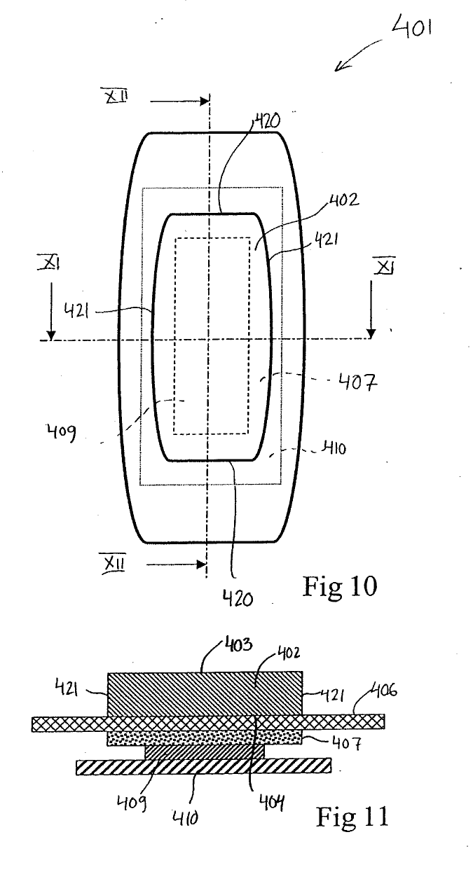

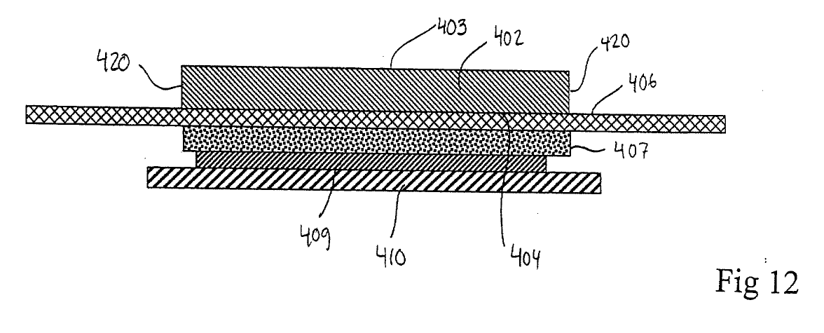

FIGS. 10-12 show an embodiment of a sanitary towel 401 of substantially the same structure as the sanitary towel 301 in FIGS. 7-9. The numbering of the various parts of the sanitary towel in FIGS. 10-12 is therefore analogous to the numbering used in the description of the sanitary towel 301 in FIGS. 7-9.

For the sanitary towel 401, the liquid barrier sheet 407 has been placed against the second separate sheet 406, on that surface which is directed away from the second surface 404 of the absorbent core. The liquid barrier sheet 407 has at least the same extent as or is greater than the absorbent core 402, which means that the second separate sheet 406 has a liquid-tight area, in at least the area of the absorbent core 402.

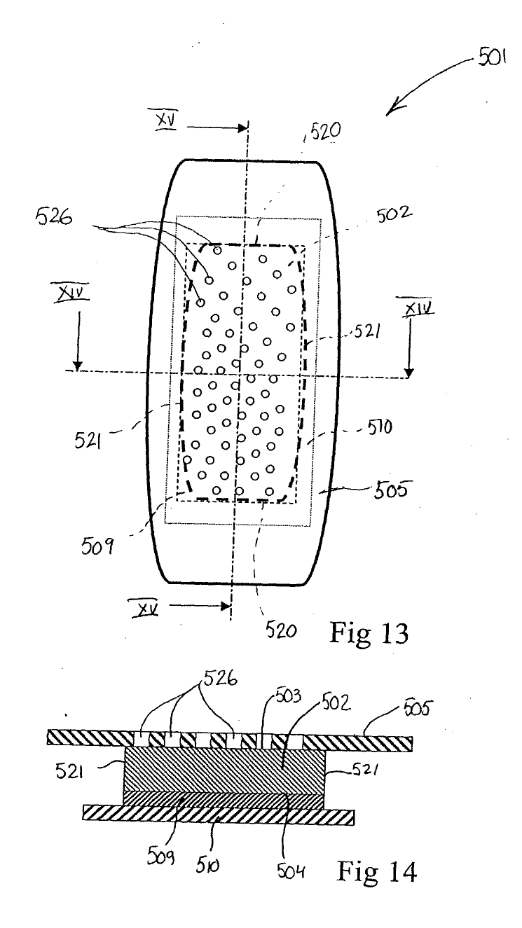

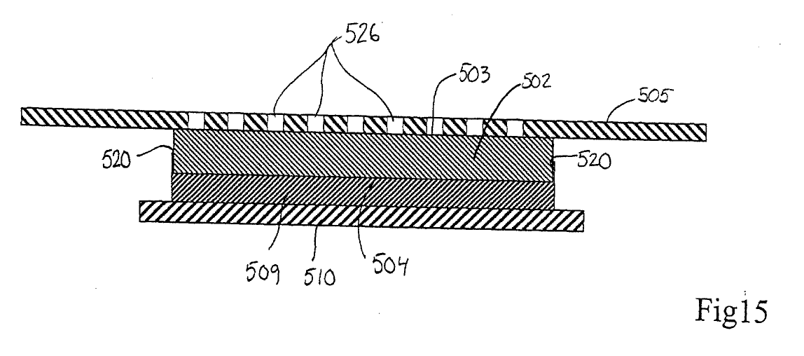

FIGS. 13-15 show an embodiment of a sanitary towel 501 of substantially the same structure as the previously described sanitary towels 101, 201, 301, 401 in FIGS. 1-12. The numbering of the various parts in FIGS. 13-15 is therefore analogous to the numbering used in the description of the sanitary towels in FIGS. 1-12.

The sanitary towel comprises an absorbent core 502, which can comprise one or more material sheets.

The absorbent core 502 comprises two surfaces, namely a first surface 503, which is directed towards the user, and a second surface 504, which is directed away from the user during use.

The sanitary towel 501 moreover comprises a first separate sheet 505 which is placed in such a way that it covers the first surface 503 of the absorbent core 502 and extends outside the longitudinal edges 521 and two transverse edges 520 of the core. The first separate sheet 505 is characterized in that it is transparent at least in the areas outside the longitudinal edges 521 and transverse edges 520 of the absorbent core 502.

The first separate sheet 505 shown in the illustrative embodiment comprises a thin, transparent, air-permeable, flexible plastic film which has been provided with a plurality of openings 526 in the area of the absorbent core 502, so that liquid reaching the sanitary towel 501 can penetrate through the first separate sheet 505 to the absorbent core 502.

Examples of suitable films are natural polypropylene and polyethylene films or laminates of these. The word natural means films without added organic or inorganic pigments, for example titanium dioxide.

To increase user comfort, the first separate sheet 505 can be treated, on the surface directed towards the user's body, with an embossed pattern for example. The first separate sheet 505 preferably has a regular transmittance in excess of 5% and a sheen of less than 25%.

For the embodiment described in FIGS. 13-15, it is preferable for the fastener 509 to cover at least the area of extent of the core 502 and the coverage it provides is so complete that liquid reaching it does not penetrate through the fastener and increase the risk of staining the user's clothes. Suitable liquid-tight fasteners 509 are, for example, foamed material, in which at least one surface has closed cells, hydrophobic adhesive, etc. When adhesive is used as a fastener 509, it is preferable for the absorbent core 502 to be completely coated, that is to say the adhesive sheet has no openings large enough to allow liquid to penetrate through the sheet.

It is of course important to choose a fastener 509 according to the requirements which have been set in respect of transparency within the area of the core. If the aim is for the sanitary towel 501 also to be transparent in the area of the absorbent core 502, the absorbent core and the fastener 509 must also have transparent properties. A fastener which has transparency is, for example, an adhesive without added coloring pigments, for example Ecomelt H 145 produced by Collano.

In those cases where the fastener 509 is an adhesive, the latter is protected by a removable protective sheet 510. The protective sheet 510 is intended to protect the adhesive fastener 509 from dirt and dust, and from adhering to other undesired surfaces or to itself, until the sanitary towel is to be used. When a velcro material or foamed material is used as a fastener 509, the protective sheet 510 can be omitted When the fastener 509 is intended to be arranged directly against the absorbent core 502, it is important that the adhesion of the fastener 509 to the user's clothes does not exceed the adhesion to the absorbent core and that the core's inner strength is sufficiently high.

If the strength between the fastener 509 and the user's clothes is greater than the inner strength of the absorbent core 502, the absorbent core 502 may be torn when the sanitary towel 501 is removed from the user's clothes. If the absorbent core 502 has a porous surface structure, it should be reinforced with a reinforcement sheet (this reinforcement sheet is not shown in the figures).

Reinforcement of the second surface 504 of the absorbent core can be obtained, for example, by providing the surface with a sheet of fibrous web comprising absorbent or nonabsorbent fibers of cotton, polyethylene, polypropylene or the like. The fibrous web can be connected to the absorbent core 502 by, for example, a mechanical pressing technique which results in the fibers of the fibrous web and of the absorbent core 502 becoming entangled in one another. A connection can also be obtained by applying energy so that a certain fusing of the fibrous web and/or fibers in the absorbent core 502 takes place, making it possible for the fibrous web to adhere to the absorbent core 502. Other examples of reinforcement sheets placed on the second surface 504 of the absorbent core are a bonded nonwoven sheet or a three-dimensional plastic sheet. Other examples of possible reinforcement sheets are bound nonwoven sheets, three-dimensional plastic sheets, tissue sheets, knitted material, woven material or the like.

If it is desired that the sanitary towel 501 has transparency in the area of the absorbent core, then, in addition to the transparent absorbent core 502, it is necessary to choose a reinforcement sheet which has transparent properties. Examples of materials for transparent reinforcement sheets are three-dimensional liquid-permeable natural plastic film or bonded fiber material comprising natural fibers of polyethylene or polypropylene. The word natural means films or fibers without added organic or inorganic pigments, for example titanium dioxide.

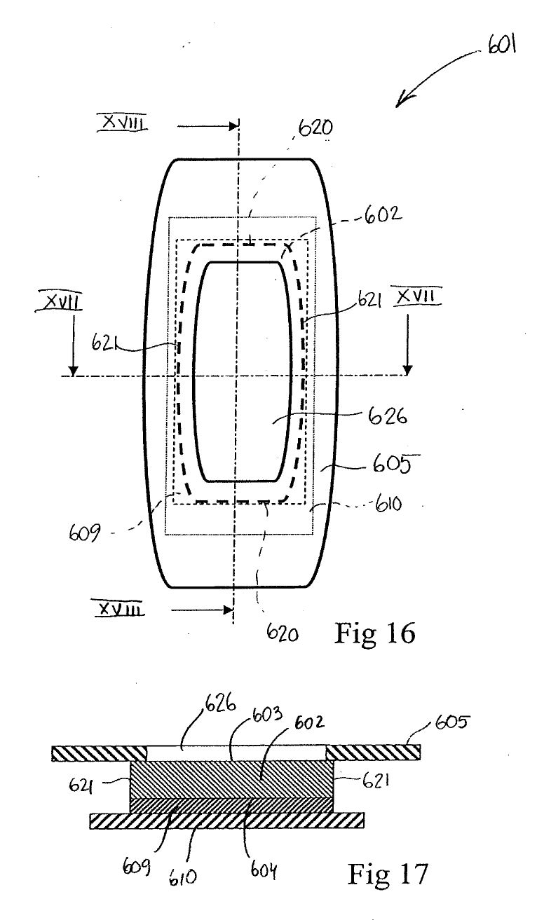

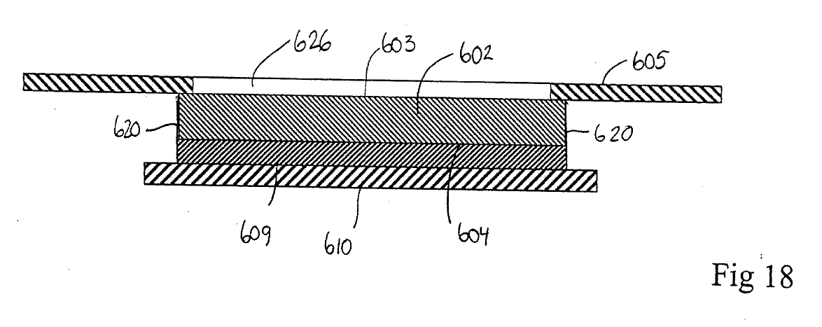

FIGS. 16-18 show an embodiment of a sanitary towel 601 of substantially the same structure as the sanitary towel 501 in FIGS. 13-15. The numbering of the various parts of the sanitary towel in FIGS. 16-18 is therefore analogous to the numbering used in the description of the sanitary towel 501 in FIGS. 13-15.

However, the sanitary towel 601 shown in FIG. 16 differs in two respects from the previously described sanitary towel.

The first separate sheet 605 consists of a thin, transparent, flexible plastic film which has been provided with a single large opening 626 in the area of the absorbent core 602, so that liquid reaching the sanitary towel 601 can be absorbed directly by the absorbent core 602. The first separate sheet 605 is transparent at least in the areas outside the longitudinal edges 621 and transverse edges 620 of the absorbent core 602.

Since the first surface 603 of the absorbent core 602 comes to bear directly against the user's body, then, if the first surface 603 of the absorbent core comprises a loosely bound structure, it is advantageous to provide the surface with a comfort layer as has been illustrated earlier in the embodiments in FIGS. 1-12.

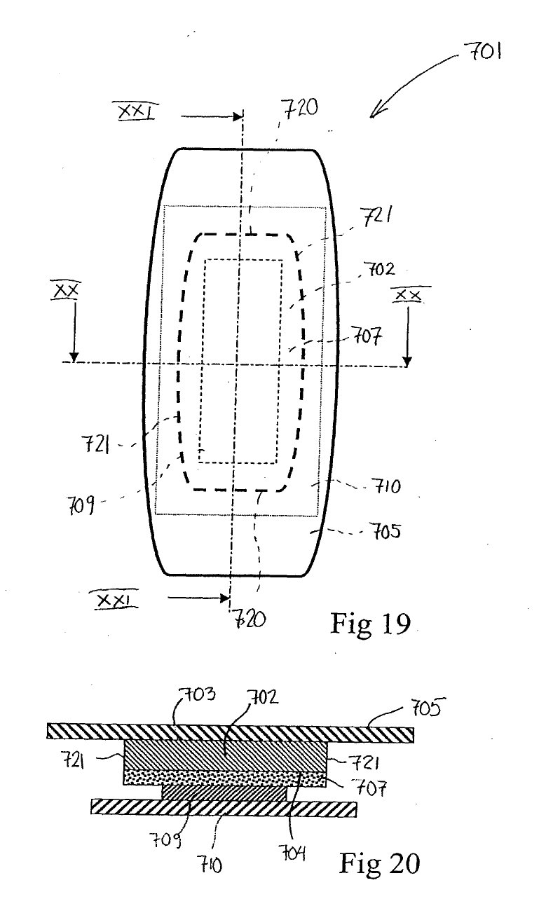

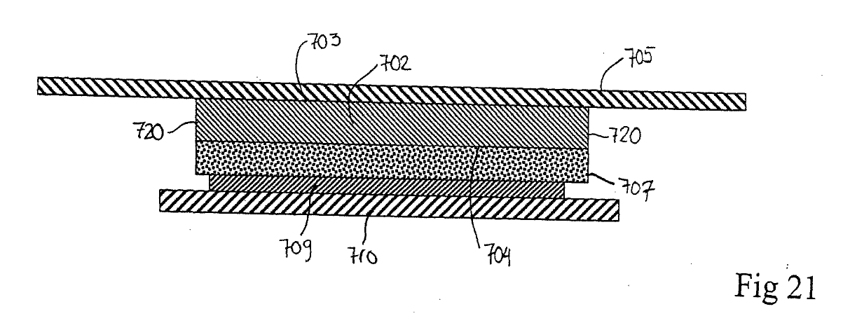

FIGS. 19-21 show an embodiment of a sanitary towel 701 of substantially the same structure as the sanitary towel 601 in FIGS. 16-18. The numbering of the various parts of the sanitary towel 701 in FIGS. 19-21 is therefore analogous to the numbering used in the description of the sanitary towel 601 in FIGS. 16-18.

However, the sanitary towel 701 shown in FIGS. 19-21 differs in two respects from the. previously described sanitary towel 601.

The first difference is that the separate sheet 705 is a liquid-permeable sheet, preferably comprising a nonwoven material. Another example of liquid-permeable material is a sheet of continuously longitudinal bound fibers. Other possible liquid-permeable materials are perforated plastic films, net, knitted or woven textiles, and combinations and laminates of the material types listed. The material can, for example, comprise natural fibers of polyethylene or polypropylene. The word natural means films or fibers without added organic or inorganic pigments, for example titanium dioxide.

The second difference between the sanitary towel 701 and the previously described sanitary towel 601 in FIGS. 16-18 is that the fastener 709 does not cover the whole area of extent of the core 702. It is therefore preferable for a liquid barrier sheet 707 to be placed on the second surface 704 of the absorbent core in order to ensure that liquid reaching the sanitary towel 701 does not spread through the sanitary towel 701 and increase the risk of staining the user's clothes. The liquid barrier sheet 707 preferably has the same extent as the absorbent core 702. The liquid barrier sheet 707 consists, for example, of hydrophobic adhesive, plastic film, foamed material with at least one surface of closed cells, hydrophilic fiber structures such as nonwovens, cellulose fiber sheets which have been treated with a hydrophobicizing means so that they have a liquid-tight surface, or similar.

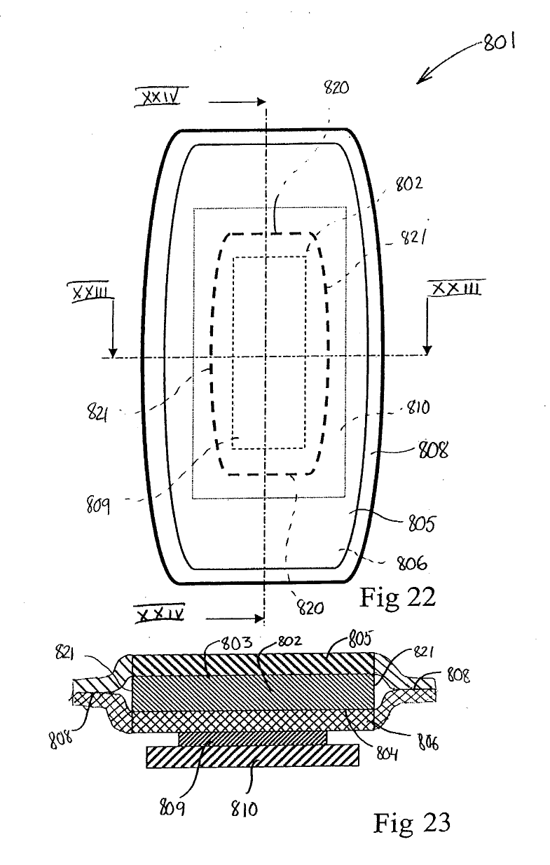

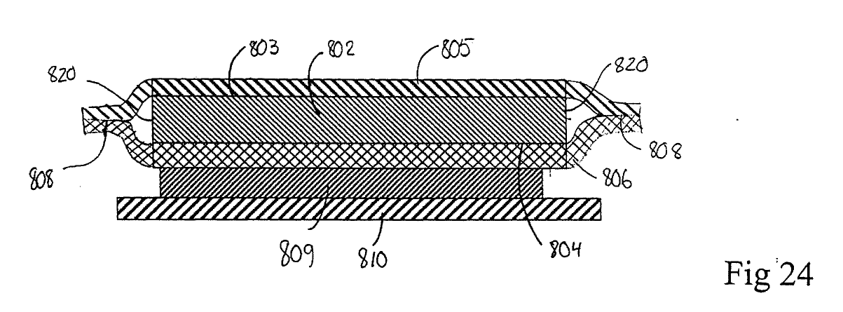

FIGS. 22-24 show an embodiment of a sanitary towel 801 of substantially the same structure as the previously described sanitary towels in FIGS. 1-21. The numbering of the various parts of the sanitary towel in FIGS. 1-21 is therefore analogous to the numbering used in the description of the sanitary towel 801 in FIGS. 22-24.

What distinguishes this embodiment is that a first separate sheet 805 is placed against the first surface 803 of the absorbent core 802 and a second separate sheet 806 is placed against the second surface 804 of the absorbent core 802. The two sheets 805, 806 cover the absorbent core 802 and extend further beyond the longitudinal and transverse edges 821, 820 of the absorbent core, the two sheets 805, 806 being joined together outside the edges of the core at a connection edge 808. The connection can be obtained by any technique known for this purpose, for example adhesive bonding, welding, sewing or the like.

The first separate sheet 805 in this embodiment is a liquid-permeable sheet which has transparent properties at least in the areas which extend beyond the longitudinal edges 821 and transverse edges 820 of the absorbent core 802. The first separate sheet 805 consists, for example, of liquid-permeable material of nonwoven fiber fabric, called nonwoven material. Another example of a liquid-permeable material is a sheet of continuously longitudinal bonded fibers. Other possible liquid-permeable materials are perforated plastic films, net, knitted or woven textiles and combinations and laminates of the material types listed. The material can for example comprise natural fibers of polyethylene or polypropylene. The word natural means films or fibers without added organic or inorganic pigments, for example titanium dioxide.

The second separate sheet 806 is transparent at least in the areas outside the longitudinal edges 821 and transverse edges 820 of the absorbent core 802. The sheet 806 consists of a thin, flexible plastic film which is liquid-tight. Examples of suitable films are natural polypropylene and polyethylene films or laminates of these. The word natural means films without added organic or inorganic pigments, for example titanium dioxide.

It is advantageous if the second separate sheet 806 is able to breathe, that is to say let through moist air (but not liquid), as this increases user comfort. The second separate sheet 806 has a regular transmittance in excess of 5% and a sheen of less than 25%.

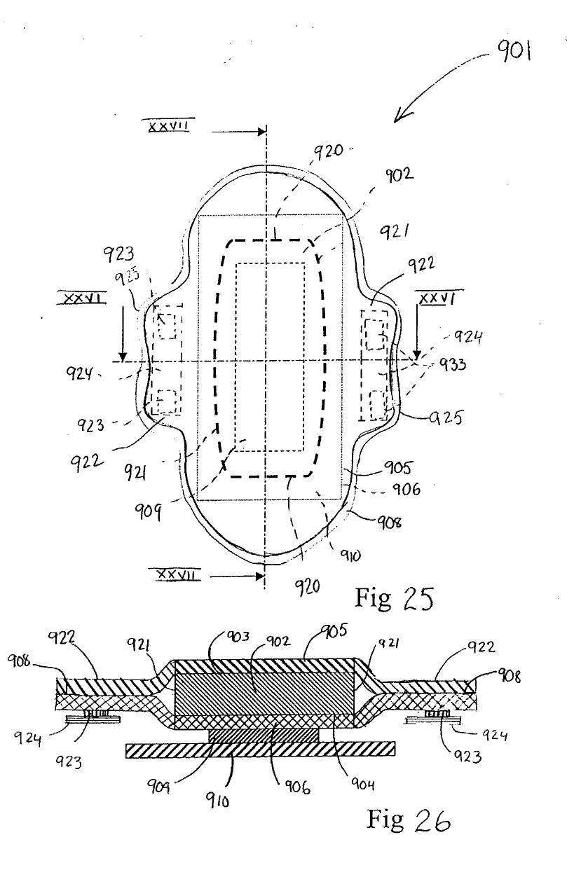

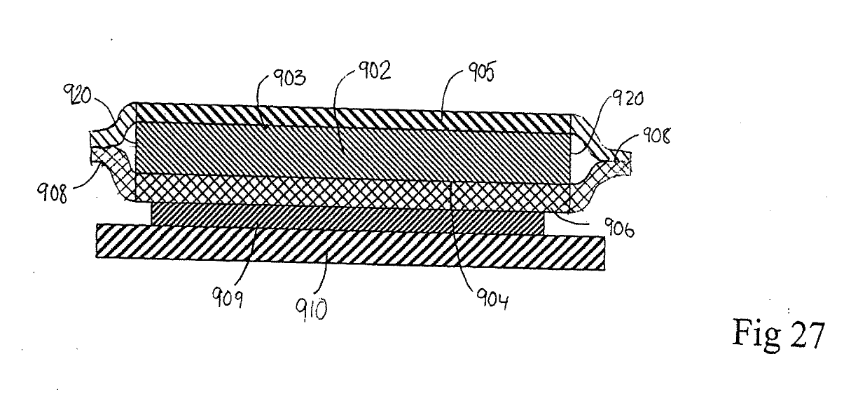

FIGS. 25-27 show an embodiment of a sanitary towel 901 of substantially the same structure as the previously described sanitary towel 801 in FIGS. 22-24. The numbering of the various parts of the sanitary towel 901 in FIGS. 25-27 is therefore analogous to the numbering used in the description of the sanitary towel 801 in FIGS. 22-24. What distinguishes this embodiment is that the first and second separate sheets 905, 906 are transparent in at least the areas which extend outside the longitudinal and transverse edges 921, 920 of the absorbent core 902. The first and second separate sheets 905, 906 in this case extend so far outside the longitudinal edges 921 of the absorbent core 902 that the sanitary towel 901 has two transparent side tabs 922. The side tabs 922 are arranged on each side of the absorbent core 902. The side tabs 922 are of such shape and size that they can be folded around the leg openings of the briefs during use.

An alternative to the embodiment described above can be that only one of the first and second separate sheets 905, 906 is used to form the transparent side tabs (not shown in the figures).

The transparent side tabs 922 of the sanitary towel 901 have been provided with a transparent fastening adhesive 923. It has been found to be expedient for the transparent side tabs 922 to have fastening adhesive 923 arranged near the outer contour line 925 of the side tabs 922 in order to create a good contact between the outer contour line 925 of the side tab 922 and the briefs, so that the side tab 922 is prevented from hanging down or being bent out from the outside of the briefs and thus becoming noticeable. Protective sheets 924 are placed over the transparent fastening adhesive 923. These protective sheets 924 are treated with release agent, at least on the surface which is arranged against the fastening adhesive 923, in order to protect the latter from dirt and dust and to prevent the adhesive from adhering to other undesired surfaces or to itself before the sanitary towel is to be used.

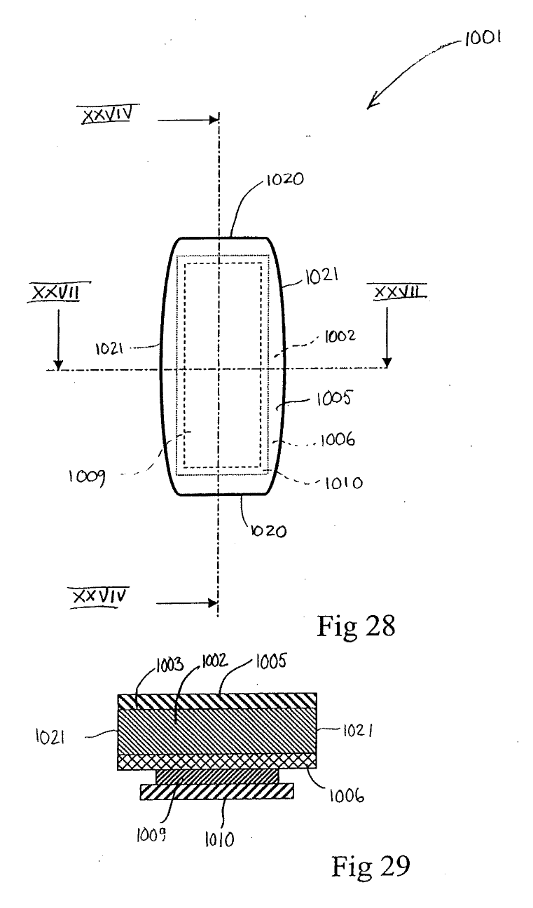

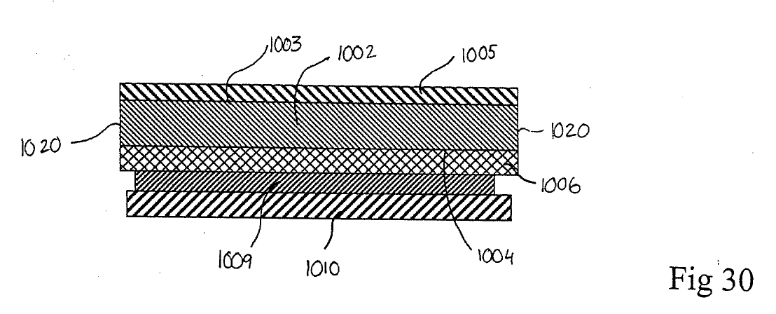

FIGS. 28-30 show an embodiment of a sanitary towel 1001 of substantially the same structure as the previously described sanitary towel 901 in FIGS. 25-27. The numbering of the various parts of the sanitary towel 1001 in FIGS. 28-30 is therefore analogous to the numbering used in the description of the sanitary towel 901 in FIGS. 25-27.

What distinguishes this embodiment is that the extent of the first and second separate sheets 1005, 1006 coincides with the longitudinal and transverse edges 1021, 1020 of the absorbent core 1002. All of the material making up the sanitary towel has transparent properties.

The sanitary towel has a regular transmittance in excess of 5% and a sheen of less than 25%.

The invention is not to be considered as being limited by the illustrative embodiments described here. All conceivable combinations and variants of the described illustrative embodiments are also intended to be covered by the scope of the invention.

What is claimed is:

1. An absorbent article, wherein at least a part of the absorbent article has a regular transmittance in excess of 5% and a sheen of less than 25%.

2. The absorbent article according to claim 1, wherein the article comprises an absorbent core with a first surface adapted to face a user's body during use, and a second surface adapted to face away from the user's body during use, and two longitudinal edges and two transverse edges and further comprising a sheet of the absorbent core that extends beyond the longitudinal edges and the transverse edges of the core.

3. The absorbent article according to claim 2, wherein at least those parts of the sheet which extend beyond the longitudinal edges and transverse edges of the absorbent core have a regular transmittance in excess of 5% and a sheen of less than 25%.

4. The absorbent article according to claim 2, wherein at least those parts of the sheet which extend beyond the longitudinal edges and transverse edges of the absorbent core have a regular transmittance in excess of 5% and a sheen of between 3 and 10%.

5. The absorbent article according to claim 2, wherein the sheet is substantially liquid-permeable.

6. The absorbent article according to claim 5, wherein the liquid-permeable sheet covers the first surface of the absorbent core.

7. The absorbent article according to claim 2, wherein the absorbent article is a sanitary towel, a panty liner or an incontinence pad.

8. The absorbent article according to claim 5, wherein the liquid-permeable sheet covers at least the second surface of the absorbent core.

9. The absorbent article according to claim 5, wherein the substantially liquid-permeable sheet is a three-dimensional natural plastic film or bonded fiber material comprising natural fibers of polyethylene or polypropylene.

10. The absorbent article according to claim 3, wherein the sheet is liquid-tight.

11. The absorbent article according to claim 10, wherein the liquid-tight sheet covers the first surface of the absorbent core.

12. The absorbent article according to claim 11, wherein at least one area in the form of at least one opening is formed in the liquid-tight sheet in the area of the absorbent core.

13. The absorbent article according to claim 10, wherein the liquid-tight sheet covers the second surface of the absorbent core.

14. The absorbent article according to claim 10, wherein the liquid-tight sheet comprises a transparent polyethylene, polypropylene film or multi-layer film comprising these materials or a breathable microporous film.

15. The absorbent article according to claim 2, wherein the sheet covers the second surface of the absorbent core and has been treated in at least an area inside the longitudinal and transverse edges of the core so that the area has an opaque surface.

16. The absorbent article according to claim 15, wherein the sheet has been pigmented with dye or embossed so that the sheet has opaque properties.

17. The absorbent article according to claim 15, wherein the sheet is provided with a separate opaque sheet that is an opaque nonwoven sheet, a plastic sheet, a cellulose sheet, or a laminate.

18. The absorbent article according to claim 16, wherein the sheet is a nontransparent fastening member.

19. The absorbent article according to claim 16, wherein the sheet is a fastening adhesive.

20. The absorbent article according to claim 2, wherein the sheet at least partially covers the first surface of the absorbent core and extends outside the longitudinal edges of the core in order to form side tabs of such shape and size that, during use of the article, the side tabs can be folded around leg edges in a crotch portion of a pair of briefs.

21. The absorbent article according to claim 2, wherein the sheet at least partially covers the second surface of the absorbent core and extends outside the longitudinal edges of the core in order to form side tabs of such shape and size that, during use of the article, the side tabs can be folded around leg edges in a crotch portion of a pair of briefs.

22. The absorbent article according to claim 20, wherein the side tabs comprise a transparent fastening member on a side of the side tabs directed towards the briefs during use.

23. The absorbent article according to claim 21, wherein the side tabs comprise a transparent fastening member on a side of the side tabs directed towards the briefs during use.

24. The absorbent article according to claim 2, wherein the absorbent article has a regular transmittance in excess of 5% in an area of the absorbent core and a sheen of less than 25% in an area of the absorbent core.

25. The absorbent article according to claim 2, wherein the sheet partially covers the first surface of the absorbent core, and a second sheet covers the second surface of the absorbent core, the two sheets being joined together outside the edges of the core at a connecting edge.

26. The absorbent article according to claim 1, wherein the article comprises an absorbent core with a first surface adapted to face a user's body during use, and a second surface adapted to face away from the user's body during use, and two longitudinal edges and two transverse edges, where at least a first or a second separate sheet at least partially covers the first surface of the absorbent core or the second surface of the core and coincides with the longitudinal edges and transverse edges of the core.

27. The absorbent article according to claim 1, wherein the article comprises an absorbent core with a first surface adapted to face a user's body during use, and a second surface adapted to face away from the user's body during use, and two longitudinal edges and two transverse edges, wherein a surface of the absorbent core extends beyond the longitudinal edges and the transverse edges of the core.
